(19) United States
(12) Patent Application Publication
(10) Pub. No.: US 2025/0345093 A1
(43) Pub. Date: Nov. 13, 2025

(54) ABSORBENT PAD INCORPORATING RECYCLED MATERIAL

(71) Applicant: U.S. Cotton, LLC, Gastonia, NC (US)

(72) Inventor: Marcel Elsener, Charlotte, NC (US)

(21) Appl. No.: 18/658,079

(22) Filed: May 8, 2024

(51) Int. Cl. A61B 17/54; A61B 17/00; A61L 15/40

(52) U.S. Cl. CPC A61B 17/54; A61L 15/40; A61B 2017/00761

(57) ABSTRACT

An absorbent pad includes: a first layer of structured fibrous material, having a central portion bounded by a peripheral edge; a second layer of structured fibrous material, having a central portion bounded by a peripheral edge; the first layer and the second layer being connected to each other near their respective peripheral edges, such that an interior space is defined between their respective central portions; and loose filler material disposed in the interior space.

### BACKGROUND OF THE INVENTION

This invention relates generally to personal care products, and more particularly to an absorbent pad incorporating recycled material.

It is known to make absorbent pads from fibrous material such as cotton. Such pads are used for tasks such as applying and removing makeup and cleansing the skin. One typical pad construction includes fibrous material compacted into a pad.

Another typical construction involves multiple layers of material which are joined or bonded together to form a pad. Multilayer pads typically have more structural integrity than single layer pads.

One problem with existing multilayer pads is that they are usually constructed of all-new materials and are manufactured using methods that waste some of the material.

### BRIEF SUMMARY OF THE INVENTION

This problem is addressed by an absorbent pad having first and second layers sandwiching unstructured material therebetween.

According to one aspect of the technology described herein, an absorbent pad includes: a first layer of structured fibrous material, having a central portion bounded by a peripheral edge; a second layer of structured fibrous material, having a central portion bounded by a peripheral edge; the first layer and the second layer being connected to each other near their respective peripheral edges, such that an interior space is defined between their respective central portions; and loose filler material disposed in the interior space.

According to another aspect of the technology described herein, a method of making an absorbent pad, includes: forming a first layer of structured fibrous material having a central portion bounded by peripheral edge; forming a second layer of structured fibrous material having a central portion bounded by peripheral edge; placing loose filler material between the respective central portions of the first and second layers; and compressing the first and the second layer together such that they are connected to each other near their respective peripheral edges.

### BRIEF DESCRIPTION OF THE DRAWINGS

The invention may be best understood by reference to the following description taken in conjunction with the accompanying drawing figures in which:

FIG. 1 is a top plan view of an absorbent pad;

FIG. 2 is a side view of the pad of FIG. 1;

FIG. 3 is a cross-sectional view of the pad of FIG. 1;

FIG. 4 is a top plan view diagram of a stack of materials for manufacturing the absorbent pad; and

FIG. 5 is a side view diagram of a manufacturing process of the pad.

### DETAILED DESCRIPTION OF THE INVENTION

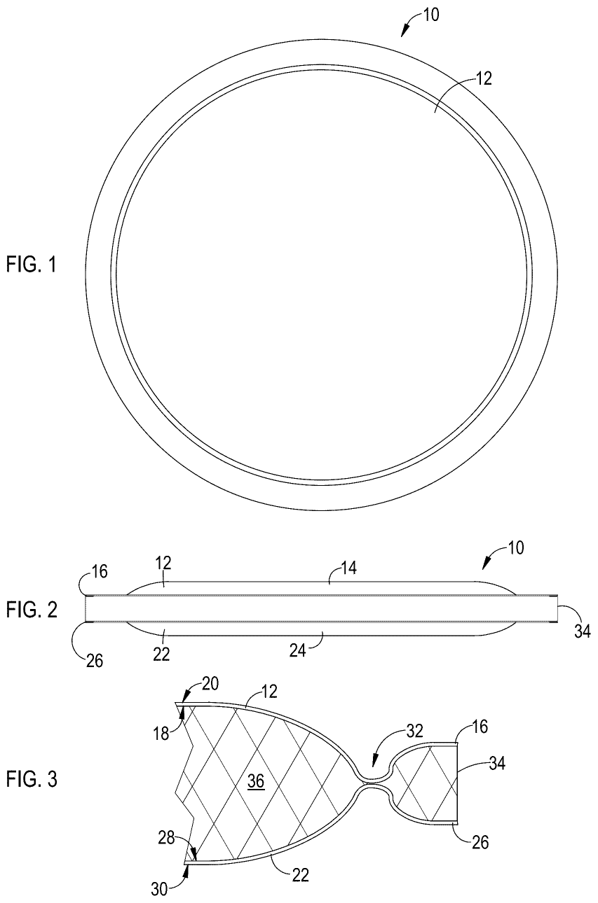

Referring to the drawings wherein identical reference numerals denote the same elements throughout the various views, FIGS. 1-3 illustrate an exemplary absorbent pad 10.

In the illustrated example, the pad 10 is a circular shape in plan view. Other shapes could be used. Nonlimiting examples of suitable shapes include ovals, squares or rectangles with or without rounded corners, other closed curves, and polygons.

The pad 10 comprises a first layer 12 having a central portion 14 bounded by a peripheral edge 16. The first layer 12 has an inner face 18 and an opposed outer face 20.

The first layer 12 comprises structured fibrous material. One exemplary material is cotton spunlace, a nonwoven material formed by entangling cotton fibers using a waterjet process. The precursor web for the spunlace may be made by various processes such as air laying or carding.

The first layer 12, in particular the outer face 20, may incorporate various surface textures. For example it may be smooth, or may include an exfoliating texture comprised of relatively protruding and relatively recessed portions, such as ridges and valleys.

The pad 10 comprises a second layer 22 having a central portion 24 bounded by a peripheral edge 26. The second layer 22 has an inner face 28 and an outer face 30.

The second layer 22 comprises structured fibrous material. One exemplary material is cotton spunlace as described above.

The second layer 22, in particular the outer face 30, may incorporate various surface textures. For example it may be smooth, or may include an exfoliating texture comprised of relatively protruding and relatively recessed portions, such as ridges and valleys.

The first layer 12 and the second layer 22 are securely connected to each other near their respective peripheral edges 16, 26.

Various means may be used for the connection. In one method, the first and second layers 12, 22 are pressed or crimped together at their peripheral edges 16, 26. It is known that the application of sufficient pressure to cellulosic fibers has a plasticizing effect which results in a secure bond. However, the hardening of the material can result in the pad presenting a sharp edge. This may be undesirable in some applications.

Alternatively, the first layer 12 and second layer 22 may be pressed or crimped together along a compression region 32 closely following but offset from their respective peripheral edges 16, 26. As shown in FIG. 3, this results in the peripheral edges 16, 26 not being bonded or connected. This allows the pad 10 to present a soft outer edge 34. The compression region may be continuous or intermittent. This type of connection may be formed by pressing the first layer 12 and the second layer 22 between appropriately configured dies. A suitable method for producing this type of connection is described in U.S. Pat. No. 7,192,630.

An interior space is defined between the central portions 14, 24 of the first and second layers 12, 22.

This interior space may be filled with a loose filler material 36, such as an unstructured fibrous material. Examples of such materials include loose fibers, or chopped or shredded fabric. For example, chopped or shredded T-shirt material (i.e. woven cotton) may be placed into the interior space. As explained in more detail below, the filler material 36 may be recycled materials.

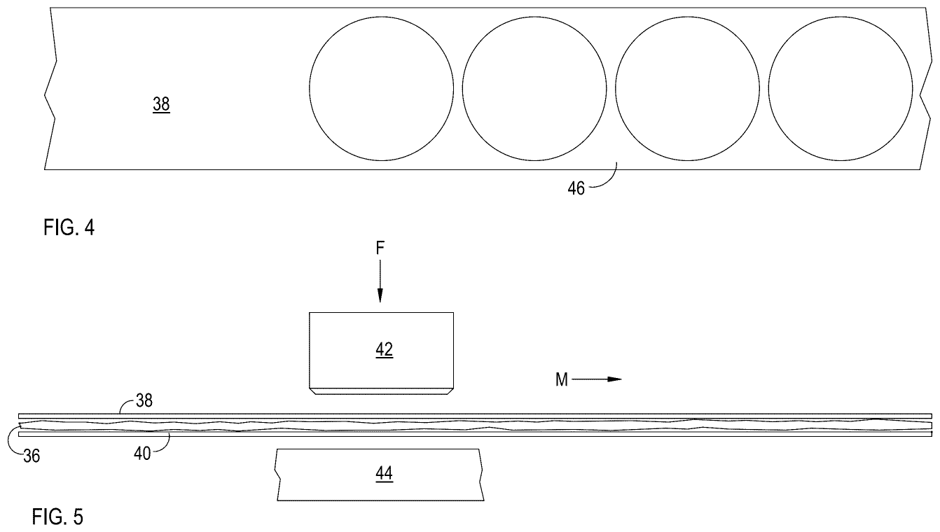

Referring to FIGS. 4 and 5, the pad 10 may be produced by stacking or superimposing a first elongated strip 38 which is a precursor for the first layer 12, a filler material 36, and a second elongated strip 40, which is a precursor for the second layer 22. The stacked materials are then fed between a male die 42 and a female die 44. The dies 42, 44 are compressed together under force F which has the effect of forming the compression region described above, connecting the first layer 12 to the second layer 22, and simultaneously or near simultaneously shearing out the pad 10 from the stack of materials.

The stacked materials are sequentially fed between the dies 42, 44 in the direction of movement marked with arrow “M” and the dies 42, 44 operated, producing a plurality of the pads 10.

As seen in FIG. 4, this process produces waste 46 in the form of the stacked materials (first and second strips 38, 40 and filler material 36), with spaces punched out in the shape of the pads 10. This waste material may be referred to as “skeletal waste”.

This material may be recycled by chopping or shredding it and then subsequently may serve as filler material 36 to manufacture more pads 10.

The process described herein provides a method for producing a high quality economical absorbent pad and reduces waste in the manufacturing process.

The foregoing has described an absorbent pad. All of the features disclosed in this specification, and/or all of the steps of any method or process so disclosed, may be combined in any combination, except combinations where at least some of such features and/or steps are mutually exclusive.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends, or to any novel one, or any novel combination, of the steps of any method or process so disclosed.

What is claimed is:

1. An absorbent pad, comprising:
a first layer of structured fibrous material, having a central portion bounded by a peripheral edge;
a second layer of structured fibrous material, having a central portion bounded by a peripheral edge;
the first layer and the second layer being connected to each other near their respective peripheral edges, such that an interior space is defined between their respective central portions; and
loose filler material disposed in the interior space.

2. The absorbent pad of claim 1, wherein the first layer and the second layer comprise cotton spunlace.

3. The absorbent pad of claim 1, wherein the filler material comprises cotton.

4. The absorbent pad of claim 1, wherein the filler material comprises chopped or shredded material.

5. The absorbent pad of claim 1, wherein the filler material comprises recycled material.

6. The absorbent pad claim 1, wherein the first and second layers are pressed or crimped together along a compression region offset from their respective peripheral edges, such that their respective peripheral edges are not bonded or connected.

7. The absorbent pad of claim 1, wherein at least one of the first and the second layers includes an exfoliating texture.

8. A method of making an absorbent pad, comprising:
forming a first layer of structured fibrous material having a central portion bounded by peripheral edge;
forming a second layer of structured fibrous material having a central portion bounded by peripheral edge;
placing loose filler material between the respective central portions of the first and second layers; and
compressing the first and the second layer together such that they are connected to each other near their respective peripheral edges.

9. The method of claim 8, wherein the first layer and the second layer comprise cotton spunlace.

10. The method of claim 8, wherein the filler material comprises cotton.

11. The method of claim 8, wherein the filler material comprises chopped or shredded material.

12. The method of claim 8, wherein the filler material comprises recycled material.

13. The method of claim 8, wherein the first layer and the second layer are formed by compressing respective elongated strips of precursor material between a pair of dies.

14. The method of claim 8, wherein the filler material is produced by:
producing skeletal waste by compressing elongated strips of precursor material between a pair of dies to shear out openings therein; and
chopping or shredding the skeletal waste.

15. The method of claim 8, further comprising:
stacking a first elongated strip which is a precursor for the first layer, the filler material, and a second elongated strip, which is a precursor for the second layer; and
feeding the stacked materials between a male die and a female die; and
compressing the dies together under force so as to shear out the first and second layers from the first and second elongated stripes and simultaneously or near simultaneously connect the first layer to the second layer.
